(19) United States
(12) Patent Application Publication
(10) Pub. No.: US 2023/0085631 A1
(43) Pub. Date: Mar. 23, 2023

(54) VISOR and HEADBAND and PROTECTIVE CONTAINER COMBINATION

(71) Applicant: Pierce Baptiste, Brooklyn, NY (US)

(72) Inventor: Pierce Baptiste, Brooklyn, NY (US)

(21) Appl. No.: 17/519,440

(22) Filed: Nov. 4, 2021

Related U.S. Application Data
(63) Continuation-in-part of application No. 29/808,589, filed on Sep. 21, 2021.

(51) Int. Cl. A42B 1/019; A42B 1/0182

(52) U.S. Cl. CPC A42B 1/019; A42B 1/0182

(57) ABSTRACT

A headwear sun visor having two fins extending from the upper extreme curvatures thereof, toward the back of the head of the wearer, combined with a detachable, size adjustable, concave headband, comprised of a twisted loops absorbent fabric material outer surface and a stretchable knitted smooth surface fabric inner surface and a reinforcement band securing the fabric materials portion to a size adjustable elastic assembly and a protective canister container.

The present application is a Continuation-In-Part of Design patent application Ser. No. 29/808,589, Title—HEADBAND and VISOR and PACKAGE combination, filing date, Sep. 21, 2021, and Design patent application Ser. No. 29/739,133, Title—HEADBAND and VISOR and PACKAGE combination, filing date Jun. 23, 2020

### BACKGROUND

### Field of Invention

The present invention is related to the field of headwear sun visors, and also to the field of athletic headbands and sports marketing, and more particularly to a headwear sun visor combined with a sweat absorbing headband and a protective container.

Athletes such as tennis players, basketball players, soccer players, volley ball players, long distance runners, and others, sweat constantly while performing. Absorbent headbands prevent sweat from entering the eyes of the wearer and hindering their performance. It is also well established that intrusive sunbeams can adversely affect the performance of athletes.

Athletic wear companies sponsor many athletic events and contract with athletes to wear their product. The need for a new product with a design element to accommodate a brand name or logo exists.

At retail, space efficient presentation and product protection is essential. A canister container allows for the headwear sun visor and adjustable headband to be neatly offered for sale.

The present invention is directed at a headwear sun visor having a design element for advertising, combined with an absorbent adjustable detachable headband and a protective canister container.

### Description of Related Art

Prior art consists of the following variations:
a) A one piece rigid cap type sun visor not covering the eyes of the wearer.
b) A face visor with headband and without design element for advertising
c) A headband without double barrier sweat protection
d) A headband without a concave surface.
e) A face visor and headband without protective canister containment.

Accordingly, a need exists for a headwear sun visor with a functional design element for advertising, combined with an adjustable headband incorporating two sweat barriers and a concave surface, combined with a protective canister container.

### SUMMARY OF THE INVENTION

In view of the foregoing, one object of the present invention is to provide a headwear sun visor combined with a detachable and adjustable headband having an absorbent twisted loops fabric outer surface and a smooth absorbent fabric inner surface, wherein when stitched together, the hidden seams of the twisted loops outer surface and the smooth absorbent fabric inner surface act as sweat barriers.

Another objective of the invention is to provide a headwear sun visor having two fins attached to and extending from each extreme upper curvature thereof, toward the back of the head of the wearer, resting above and parallel to the sweatband when in use. Said fins made from fabric material or non-fabric material suitable for application of corporation brand names and logos by printing, embroidery or other means, such a woven cotton, woven linen, woven polyester, or any blend thereof, or any vinyl coated woven fabric material, or yielding polymer material.

Yet another objective of the invention is to provide a headband that is absorbent, size adjustable and reinforced at the joint between the twisted loops fabric outer surface, the smooth inner surface, and the elastic adjustable strap, by a band stitched a both edges.

A further objective of the invention is to provide a cost effective package for the headwear sun visor and headband combination.

### BRIEF DESCRIPTION OF THE DRAWINGS

FIG. 1 is a top plan view of the twisted loops absorbent fabric outer surface frontal portion of the headband.

FIG. 2 is a twisted loop removed from the surface of FIG. 1

FIG. 3 is a top plain view of the smooth absorbent fabric inner surface frontal portion of the headband

FIG. 4 is a top plain view of the folded underside of the reinforcement band.

FIG. 5 is a top plain view of left side fins 51.

FIG. 6 is a top plain view of right side fin 61

FIG. 7 is a top plain view of the elastic assembly right side portion.

FIG. 8 is a top plain view of the elastic assembly left side portion.

FIG. 9 is a top plain view of buckle 1

FIG. 10 is a top plain view of buckle 2

FIG. 11 a top plain view of the female snap sets 31 and 41. Each set comprise of the female socket and the retention cap.

FIG. 12 is a top plain view of the male snap sets 32 and 42. Each set comprise of the male post and the retention cap.

FIG. 13 is an enlarged view of the female socket portion of FIG. 11.

FIG. 14 is an enlarged view of the male post of FIG. 12.

FIG. 15 is a top plain view of FIG. 1 and FIG. 3 stitched together and folded with the stitched seams hidden within both layers and the surface of FIG. 1 visible.

FIG. 16 is a view of FIG. 15 with the surface of FIG. 3 visible.

FIG. 17 is a top plan view of the right side of the headband with female snap 31

FIG. 18 is a top plan view of the left side of the headband with female snap 41

FIG. 19 is a front elevational view of the frontal portion of the headband.

FIG. 20 is a top plan view of the visor without fins attached.

FIG. 21 is a top plain view of the visor with fins 51 and 61 attached with male snaps 32 and 42

FIG. 22 is a reverse side view of FIG. 21

FIG. 23 is a right side elevational view of the present invention.

FIG. 24 is a left side elevational view of the present invention.

FIG. 25 is a side elevational view of FIG. 23 with the visor lifted over the headband.

FIG. 26 is a front elevational view of the present invention

FIG. 27 is a back elevational view of FIG. 26

FIG. 28 is an elevational view of the protective carrying canaster with the visor partially enclosed.

FIG. 29 is an elevational view of the protective carrying canaster with the visor and headband partially enclosed.

FIG. 30 is an elevational view of the protective carrying canaster with the visor and headband enclosed.

FIG. 31 is a side elevational view of the headband without the visor, with a decorated fin attached in place of the visor.

### DESCRIPTION OF THE PREFERRED EMBODIMENT

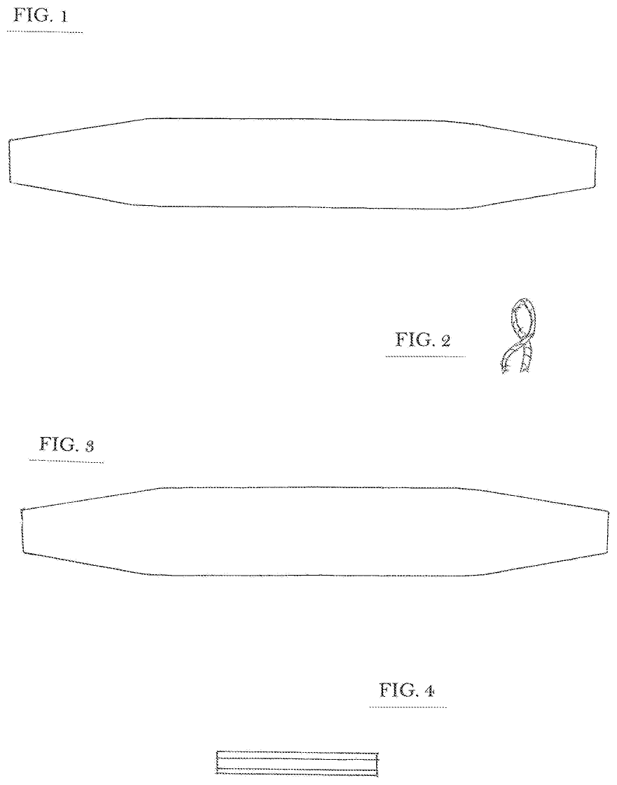

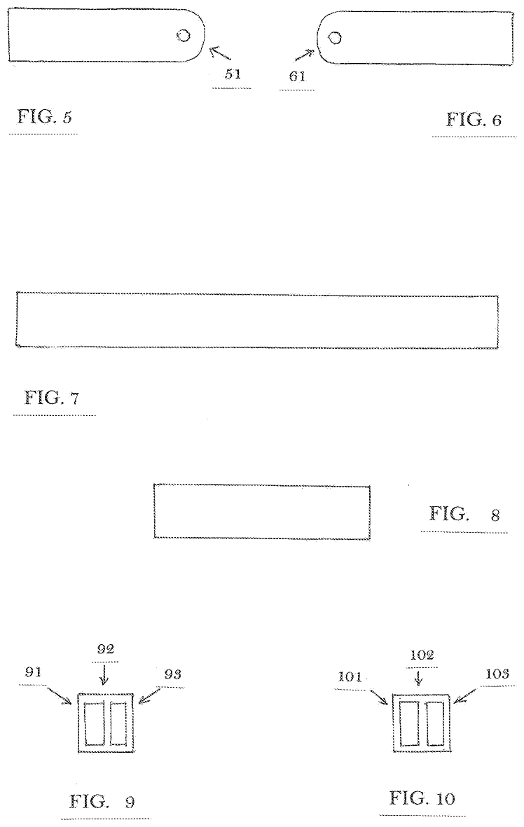

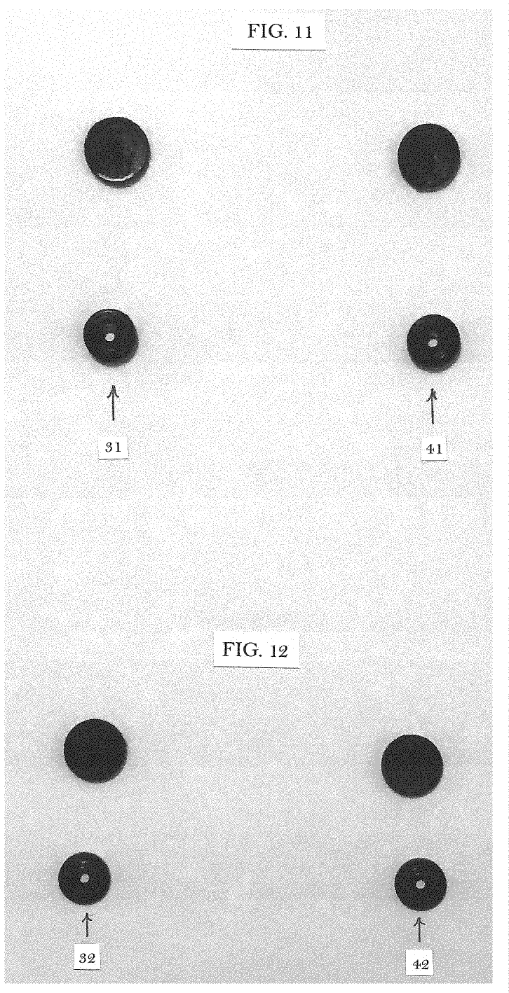

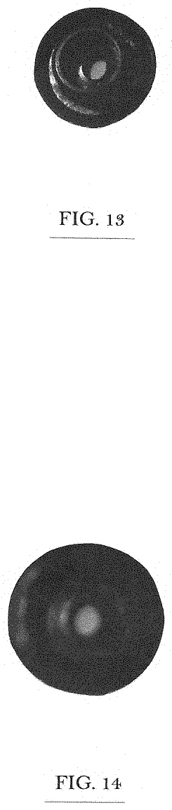

As shown in FIG. 1, a fabric panel having twisted loops woven from absorbent yard on the top and bottom surfaces thereof, is shaped symmetrically with ends narrower than it's center. FIG. 3 a knitted, stretchable, absorbent, smooth surface fabric is shaped identical to the form of FIG. 1. FIG. 1 and FIG. 2 are stitched together, desirable surfaces facing each other, along the two long edges, leaving two openings at the narrow ends. The resulting tubelike form is then turned inside out so that the stitched seams are hidden; forming a concave apex when folded and like surfaces meet, FIG. 15 and FIG. 16.

The elastic adjustment assembly is formed by threading the right side end of the elastic portion FIG. 7, over and around column 92 of buckle 9 and stitched to itself so as to form a closed loop around 92. The left side portion of FIG. 7 is threaded through buckle FIG. 10, under and over column 102, under column 103, and returning to FIG. 9 under column 91, over column 92 and under shoulder 93. The end of same left side of FIG. 7 is extended beyond buckle shoulder 93 for attachment to the frontal portion of the headband. The right side end of FIG. 8 is looped over shoulder 101, FIG. 10 and stitched to itself to form a closed loop around 101.

Elastic assembly left side portion FIG. 8 end, as assembled, is inserted into the left side opening of tubelike form FIG. 15 (FIG. 16), and top stitched together. The reinforcement band FIG. 4 is placed folded side down overlaying the joint of tubelike FIG. 15 (FIG. 16), and the elastic portion FIG. 8 end, as assembled, and top stitched at both edges so that one edge of reinforcement band FIG. 4 is stitched over the elastic portion, FIG. 8 end, as assembled, and the other edge of reinforcement band FIG. 4 is stitched over the twisted loops tubelike FIG. 15 (FIG. 16).

Elastic assembly left side portion FIG. 7 end, as assembled, is inserted into right side opening of the tubelike form FIG. 15 (FIG. 16) and the afore mentioned steps for attachment of FIG. 8 end, as assembled, is repeated.

Considering the headband as a whole, buckle 9 is allowed horizontal movement along elastic back left side portion FIG. 7, thereby providing means of adjusting the size of the headband.

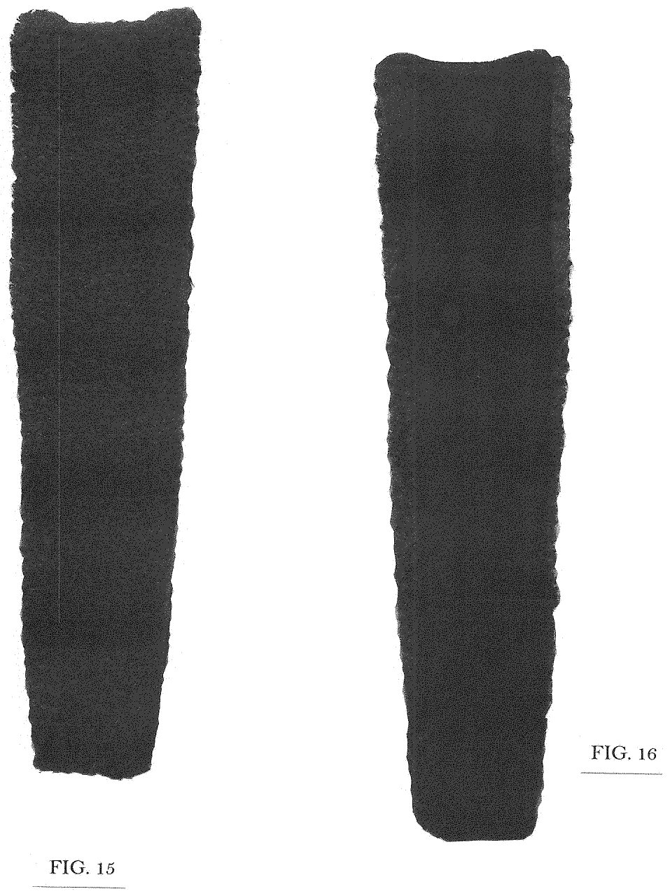

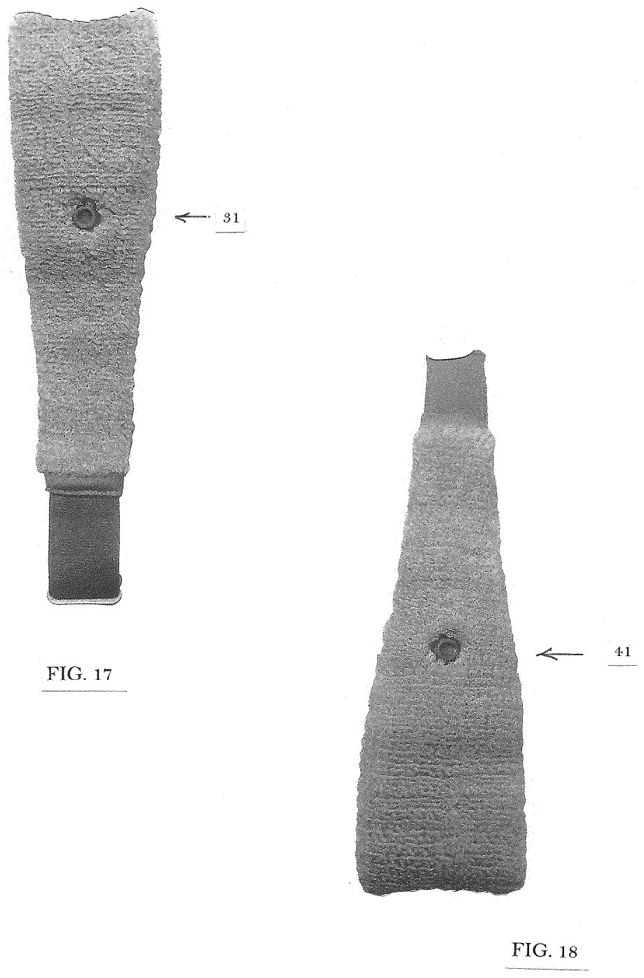

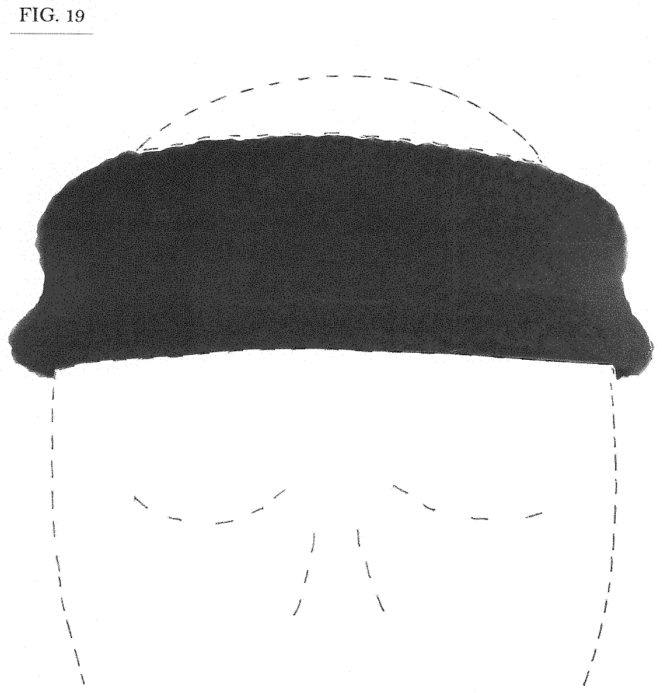

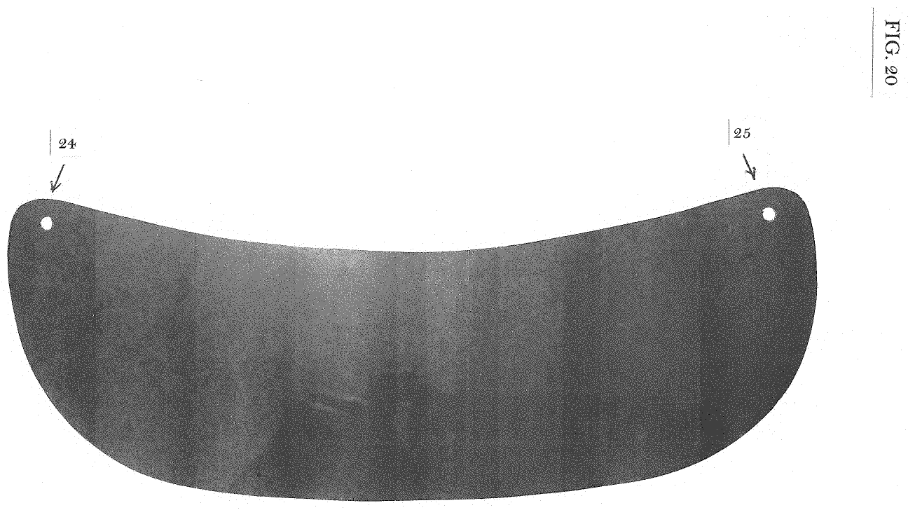

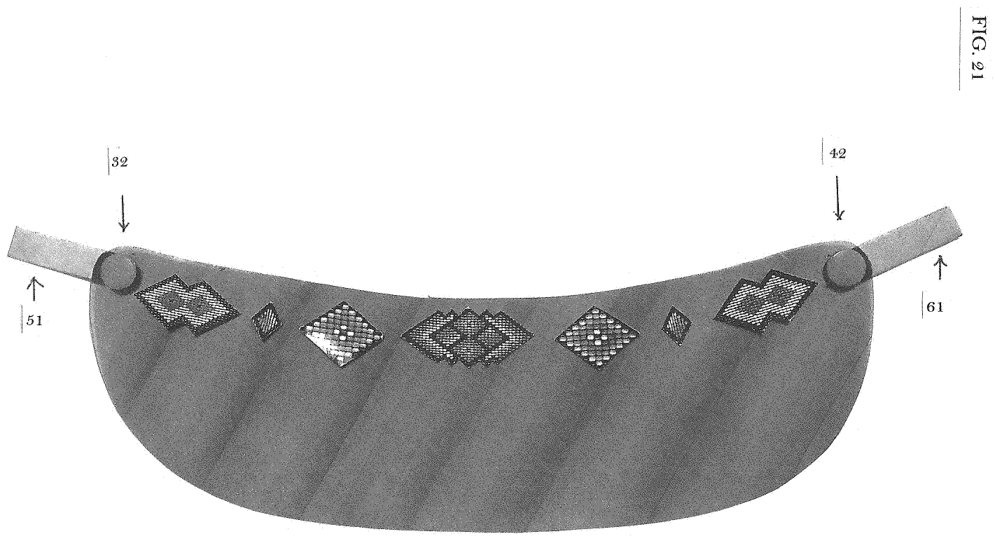

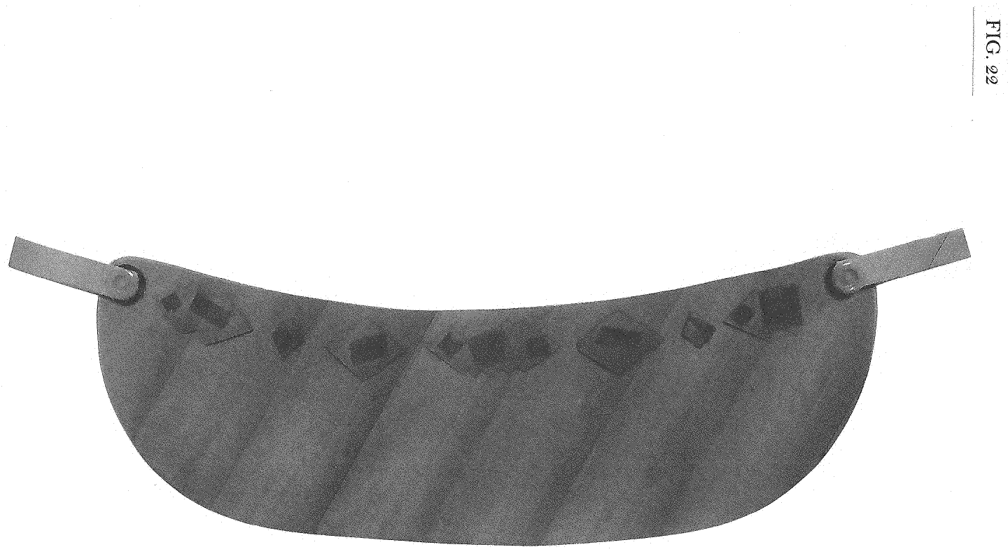

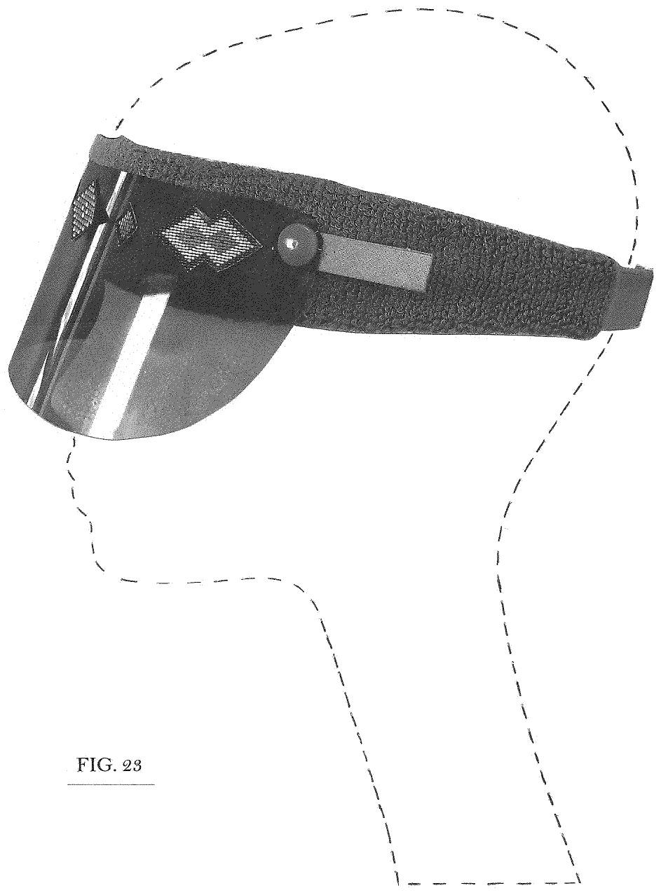

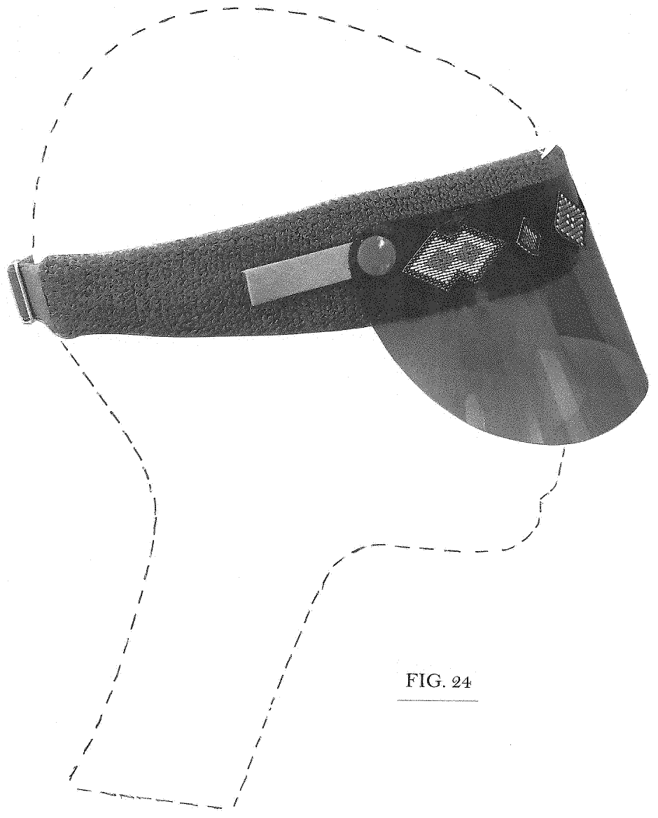

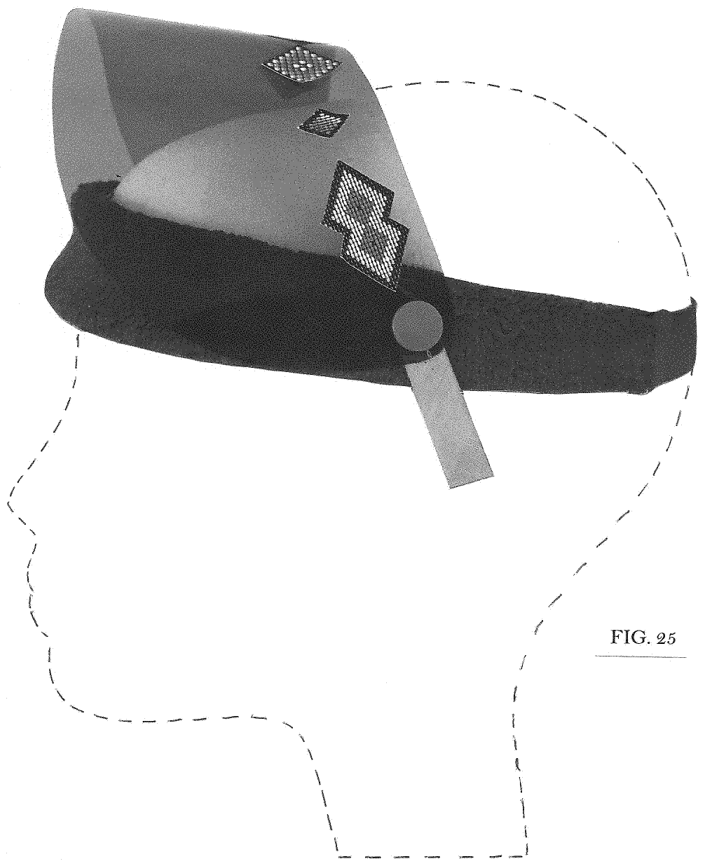

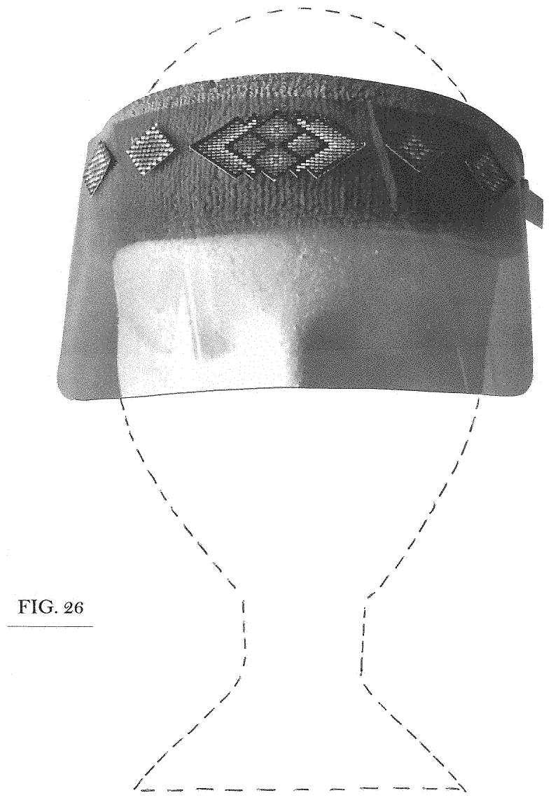

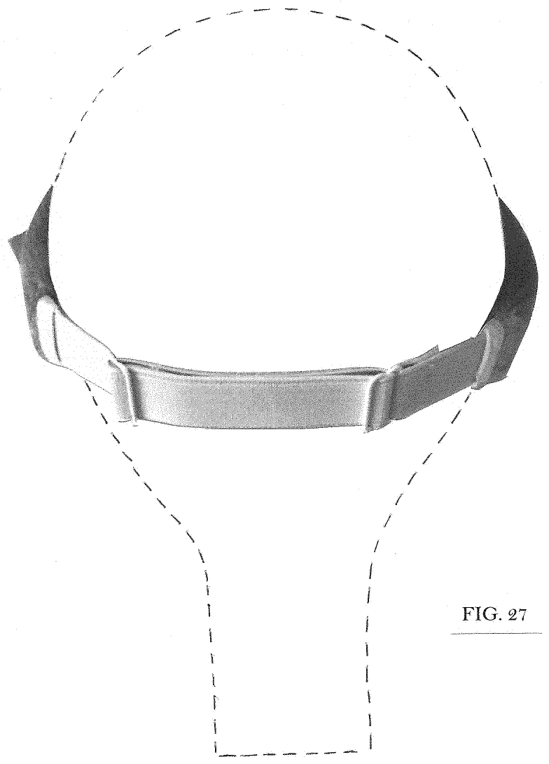

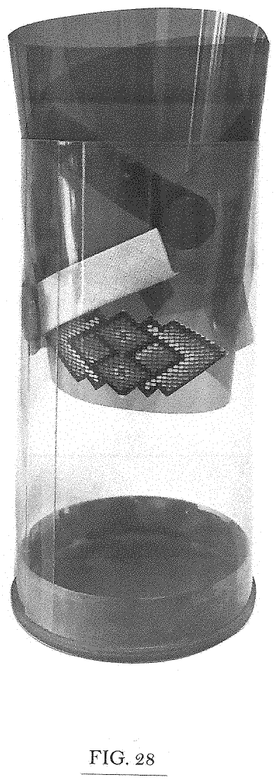

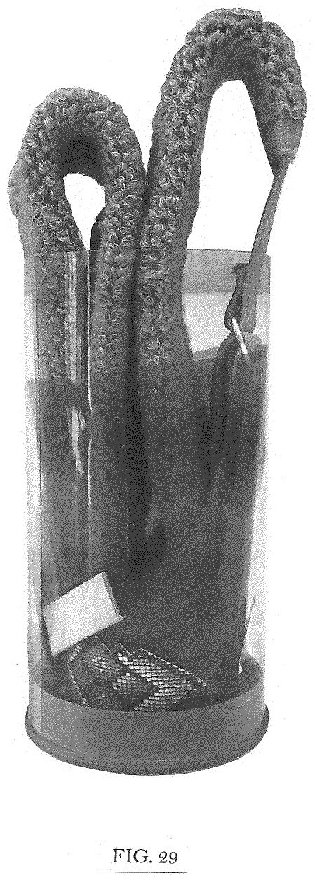

Female snaps 31 and 41 are attached to the twisted loops absorbent fabric and knitted smooth surface absorbent fabric portion of the headband, FIG. 17 and FIG. 18

The headband assembly is the integration of four parts; namely, the twisted loops absorbent fabric material, the stretchable knitted smooth surface fabric material, the folded edge reinforcement band, and the elastic unit for adjusting the size of the headband.

Fin 51 and Fin 61 are made from a durable printable fabric or non-fabric material. Visor FIG. 20, 21, 22 is made from a yielding polymer material with ultra violet rays protection characteristics.

Fins 51 is attached to the underside of visor FIG. 20 with male snap 32 fastened through hole 24. Fin 61 is attached to the underside of visor FIG. 20 with male snap 42 fastened through hole 25.

In use, the headwear sun visor portion is attached to the headband portion at the points where fin 51 and fin 61 are attached to the visor 20.

The headband disclosed herein, without the visor part, with fins attached to female snaps 31 and 41 of FIG. 17 and FIG. 18, is considered within the scope of this invention as shown in FIG. 31.

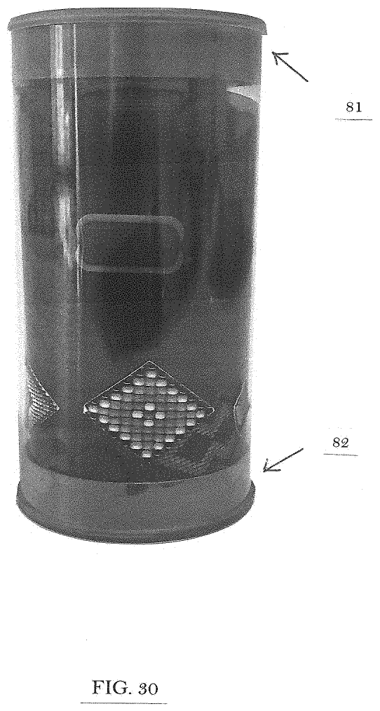

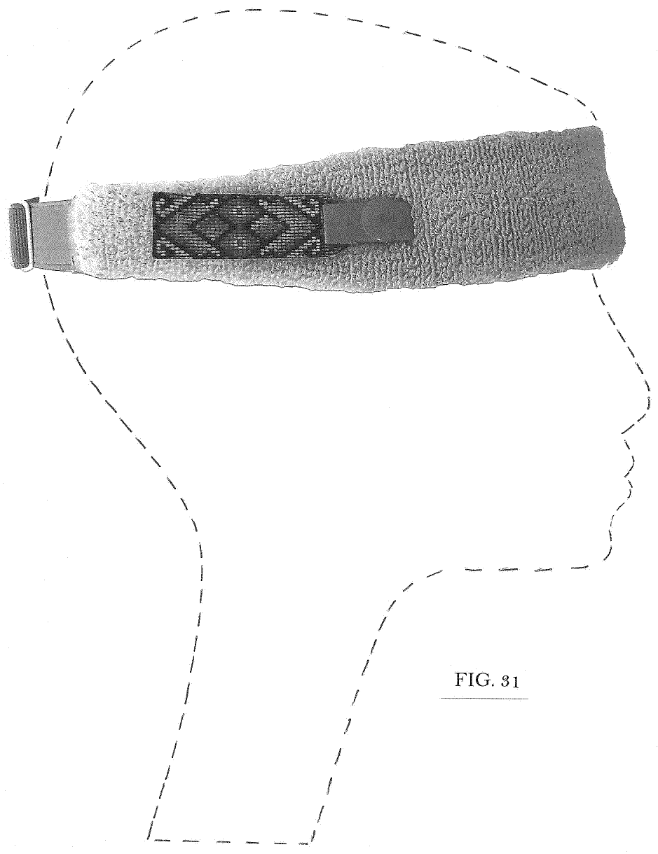

The canister protection container package is a polymer material tube FIG. 30 with caps 81 and 82.

I claim:

1) A headwear sun visor and headband and protective canister container combination; comprising of a sun visor having two fins at the upper extreme curvatures thereof, combined with a four part size adjustable headband assembly, and a protective canister packaging container.

2) The headwear sun visor and headband as set forth in claim 1, wherein the two fins extend from the point of attachment to the visor toward the back of the head of the wearer.

3) The headwear sun visor and headband as set forth in claim 2, wherein the headband is comprised of a twisted loops absorbent fabric material outer surface, and a stretchable knitted smooth fabric material inner surface, and a reinforcement band, and an elastic assembly to facilitate size adjustment.

4) The headwear sun visor and headband as set forth in claim 3, wherein the headwear sun visor is detachable from the headband.

5) The headwear sun visor and headband as set forth in claim 4, wherein the headband consists of a concealed upper seam and a concealed lower seam within the frontal portion of the headband.

6) The headwear sun visor and headband as set forth in claim 5, wherein the headband concealed upper seam and the concealed lower seam across the forehead of the wearer form a concave surface between the two seams.

7) The headwear sun visor and headband as set forth in claim 6, wherein the two fins are made from woven cotton, or woven linen, or woven polyester, or any blend thereof.

8) The headwear sun visor and headband as set forth in claim 7, wherein the woven material is vinyl-coated.

9) The head wear sun visor and headband as set forth in claim 6, wherein the two fins are made from a non fabric yielding polymer material.

10) The headband of claim 6 with a decorative fin and without a headwear sun visor.
